(19) United States
(12) Patent Application Publication
(10) Pub. No.: US 2018/0207407 A1
(43) Pub. Date: Jul. 26, 2018

(54) GUIDE WIRE AND MANUFACTURING METHOD OF GUIDE WIRE

(71) Applicant: TERUMO KABUSHIKI KAISHA, Tokyo (JP)

(72) Inventor: Nobuyuki TANIGAKI, Tokyo (JP)

(73) Assignee: TERUMO KABUSHIKI KAISHA, Tokyo (JP)

(21) Appl. No.: 15/816,172

(22) Filed: Nov. 17, 2017

(30) Foreign Application Priority Data
Jan. 20, 2017 (JP) 2017-008869

(51) Int. Cl. A61M 25/09; B21F 45/00

(52) U.S. Cl. CPC A61M 25/09; B21F 45/008; A61M 2025/09075; A61M 2025/09108; A61M 2025/09166

(57) ABSTRACT

A guide wire has an elongated core portion, a resin coating layer including a resin material and covering a distal portion of the core portion, and a metallic tubular member that has a lumen into which the core portion is inserted, and that is disposed so as to be in contact with at least a portion of a proximal end of the resin coating layer. The tubular member is formed in a state where the lumen is reduced in diameter by cold forging, and at least a portion of an inner surface forming the lumen has a compressive bonding surface which is compressively bonded to an outer surface of the core portion.

### CROSS-REFERENCES TO RELATED APPLICATIONS

This application claims the benefit of Japanese Application No. JP2017-008869 filed on Jan. 20, 2017, the entire content of which is incorporated herein by reference.

### TECHNICAL FIELD

The present invention relates to a guide wire and a manufacturing method of a guide wire.

### BACKGROUND ART

Known treatment methods involving a catheter device inside a body lumen use a guide wire to guide the catheter device to a target site of the body lumen. Known guide wires for this purpose have a resin coating layer disposed in a distal portion of a core portion (core wire) in order to improve safety and sliding performance for the body lumen or the catheter. When this guide wire is used inside other devices such as the catheter, a proximal portion of the resin coating layer may be separated and stripped off from the core portion, in some cases.

For example, International Application Publication No. WO2013/100045 discloses a guide wire in which a tubular member is disposed on a proximal side of the resin coating layer in order to prevent the proximal portion of the resin coating layer from being stripped off. A core portion is inserted into a lumen of the tubular member so that the tubular member is disposed on the proximal side of the resin coating layer.

### SUMMARY

However, if the guide wire is bent when the guide wire passes through the inside of a curved or meandering body lumen, the tubular member may pull away from the core portion. Consequently, there is a possibility that the tubular member may slip out of the core portion, or that an end portion of the tubular member may protrude to the catheter side and may get caught on the catheter. A guide wire and a manufacturing method of a guide wire according to the present disclosure can prevent a resin coating layer from being separated and stripped off from a core portion, and can prevent a tubular member from slipping out of the core portion or getting caught on a catheter.

A guide wire according to the present disclosure has an elongated core portion, a resin coating layer that is made of a resin material, and that covers a distal portion of the core portion, and a metallic tubular member that has a lumen into which the core portion is inserted, and that is disposed so as to be in contact with at least a portion of a proximal end of the resin coating layer. The tubular member is formed in a state where the lumen is reduced in diameter by cold forging, and at least a portion of an inner surface forming the lumen has a compressive bonding surface which is compressively bonded to an outer surface of the core portion.

A manufacturing method of a guide wire which has an elongated core portion, a resin coating layer made of a resin material so as to cover a distal portion of the core portion, and a metallic tubular member according to the present disclosure has a step of inserting the core portion into a lumen of the tubular member, and disposing the tubular member so as to be in contact with at least a portion of a proximal end of the resin coating layer, and a cold forging step of reducing a diameter of the lumen of the tubular member by cold forging, and forming a compressive bonding surface compressively bonded to an outer surface of the core portion in at least a portion of an inner surface forming the lumen.

According to the guide wire configured as described above, the tubular member is disposed so as to be in contact with at least a portion of the proximal end of the resin coating layer. Accordingly, the resin coating layer can be prevented from being separated and stripped off from the core portion. In addition, the tubular member includes the compressive bonding surface. Accordingly, the tubular member can be restrained from pulling away from the core portion. In this manner, the tubular member can be prevented from slipping out of the core portion or getting caught on the catheter.

According to the manufacturing method of the guide wire configured as described above, the tubular member is disposed so as to be in contact with at least a portion of the proximal end of the resin coating layer. Accordingly, the resin coating layer can be prevented from being separated and stripped off from the core portion. In addition, the diameter of the lumen of the tubular member is reduced by cold forging, and the compressive bonding surface compressively bonded to the outer surface of the core portion is formed on the inner surface of the tubular member. Accordingly, the tubular member can be restrained from pulling away from the core portion. In this manner, the tubular member can be prevented from slipping out of the core portion and getting caught on the catheter.

### BRIEF DESCRIPTION OF THE DRAWINGS

FIG. 1 is an axial cross-sectional view of a guide wire according to a first embodiment.

FIG. 2 is an axial cross-sectional view illustrating an enlarged main portion of the guide wire according to the first embodiment.

FIGS. 3(A) to 3(D) are views schematically illustrating a manufacturing method of the guide wire according to the first embodiment.

FIG. 4 is a schematic view for describing swaging.

FIGS. 5(A) and 5(B) are views schematically illustrating a manufacturing method of a guide wire according to a comparative example.

FIG. 6(A) is an axial cross-sectional view illustrating a guide wire according to a comparative example, and FIG. 6(B) is an axial cross-sectional view illustrating a state where the guide wire illustrated in FIG. 6(A) is bent.

FIG. 7(A) is an axial cross-sectional view illustrating the guide wire according to the first embodiment, and FIG. 7(B) is an axial cross-sectional view illustrating a state where the guide wire illustrated in FIG. 7(A) is bent.

FIG. 8 is an axial cross-sectional view illustrating an enlarged main portion of a guide wire according to a second embodiment.

FIG. 9(A) is an axial cross-sectional view of a core portion according to the second embodiment, and FIG. 9(B) is a cross-sectional view taken along the line 9B-9B illustrated in FIG. 9(A).

FIGS. 10(A) to 10(D) are views schematically illustrating a manufacturing method of the guide wire according to the second embodiment.

FIG. 11(A) is an axial cross-sectional view of a core portion according to Modification Example 1 of the second embodiment, and FIG. 11(B) is a cross-sectional view taken along line 11B-11B illustrated in FIG. 11(A).

FIG. 12 is a side view illustrating a core portion according to Modification Example 2 of the second embodiment.

FIG. 13 is a side view illustrating a core portion according to Modification Example 3 of the second embodiment.

FIG. 14 is a side view illustrating a core portion according to Modification Example 4 of the second embodiment.

FIG. 15 is an axial cross-sectional view illustrating an enlarged main portion of a guide wire according to a third embodiment.

FIGS. 16(A) to 16(C) are views schematically illustrating a manufacturing method of the guide wire according to the third embodiment.

FIG. 17 is an axial cross-sectional view illustrating an enlarged main portion of a guide wire according to Modification Example 1 of the third embodiment.

FIG. 18 is an axial cross-sectional view illustrating an enlarged main portion of a guide wire according to Modification Example 2 of the third embodiment.

FIG. 19 is an axial cross-sectional view illustrating an enlarged main portion of a guide wire according to an alternative example.

FIG. 20 is an axial cross-sectional view illustrating an enlarged main portion of a guide wire according to another alternative example.

FIG. 21 is an axial cross-sectional view illustrating an enlarged main portion of a guide wire according to yet another alternative example.

### DETAILED DESCRIPTION

Hereinafter, embodiments according to the present disclosure will be described with reference to the accompanying drawings. The following description does not limit the technical scope or the meaning of the terms disclosed in claims. In addition, dimensional proportions in the drawings may be exaggerated and different from actual proportions for convenience of description in some cases.

### First Embodiment

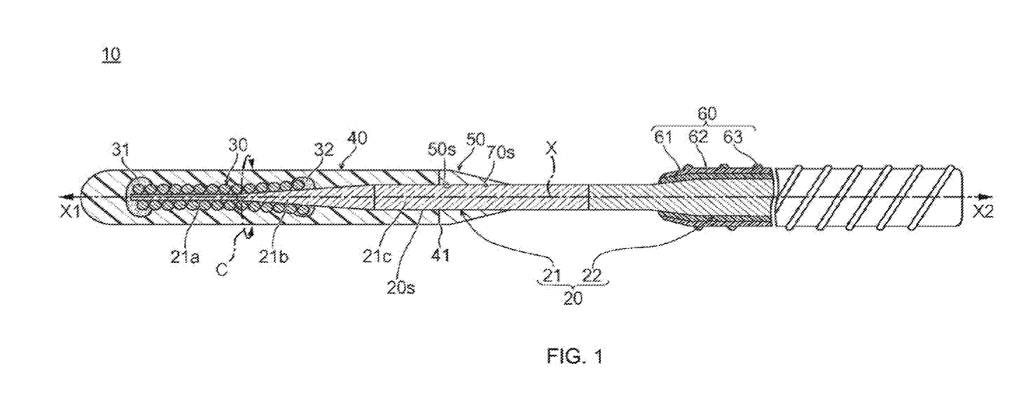

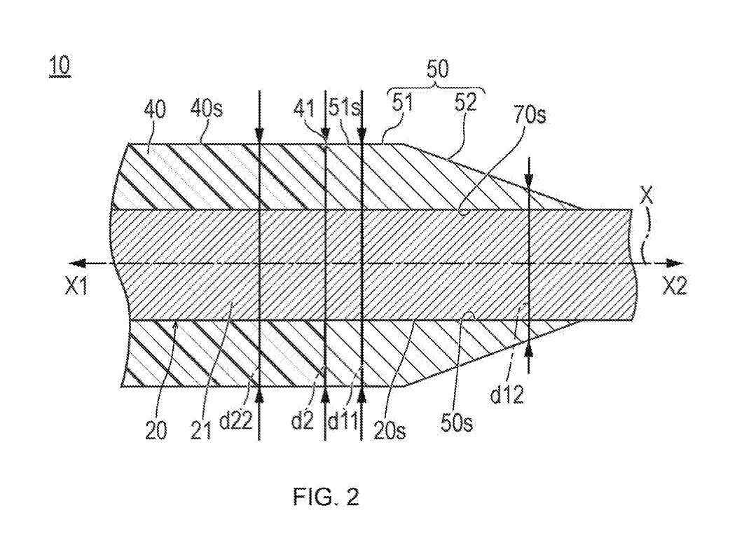

FIG. 1 is an axial cross-sectional view of a guide wire 10 according to a first embodiment, and FIG. 2 is an axial cross-sectional view illustrating an enlarged main portion of the guide wire 10.

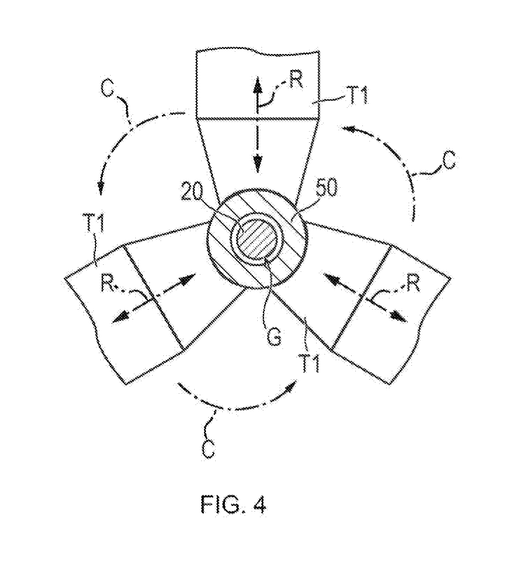

In the description herein, a longitudinal direction (leftward-rightward direction in FIG. 1) of a core portion 20 of the guide wire 10 is defined as an axial direction, and is indicated by an arrow X in each drawing. In addition, a direction rotating around the axis of the core portion 20 is defined as a circumferential direction, and is indicated by an arrow C in each drawing. In addition, a direction orthogonal to the axial direction is defined as a radial direction, and is indicated by an arrow R in FIG. 4. In addition, a side inserted into a living body (into a blood vessel) in the guide wire 10 is defined as a distal side (distal end side, left side in FIG. 1), and is indicated by an arrow X1 in each drawing. A hand operating side located opposite to the distal side is defined as a proximal side (proximal end side, right side in FIG. 1), and is indicated by an arrow X2 in each drawing. In addition, a distal portion in the description herein means a portion including a prescribed range from a distal end (most distal end) in the axial direction, and a proximal portion means a portion including a prescribed range from a proximal end (most proximal end) in the axial direction.

For example, in a state where the guide wire 10 is inserted into a lumen (guide wire lumen) of a treatment-purpose or diagnosis-purpose catheter inserted into a body lumen, the guide wire 10 is used in order to guide the catheter to a target site of the body lumen. As illustrated in FIG. 1, the guide wire 10 according to the first embodiment includes the core portion (core wire) 20 which extends in the axial direction, a coil portion 30 and a distal side coating layer (corresponding to a resin coating layer) 40 which are disposed in the distal portion of the core portion 20, a tubular member 50 which is disposed on the proximal side of the distal side coating layer 40, and a proximal side coating layer 60 which is disposed in proximal portion of the core portion 20.

A length of the guide wire 10 along the axial direction is not particularly limited. However, the length can be 200 to 5,000 mm, for example.

(Core Portion)

As illustrated in FIG. 1, the core portion 20 has a first core portion 21 which is disposed on the distal side in the axial direction, and a second core portion 22 which is disposed on the proximal side of the first core portion 21, and which is joined to the first core portion 21.

The first core portion 21 has a round bar shaped distal portion 21a which is disposed on the distal side and which has a circular cross-sectional shape, a tapered portion 21b which extends from the distal portion 21a to the proximal side, and a constant outer diameter portion 21c which extends from the tapered portion 21b to the proximal side along the axial direction while maintaining substantially a constant outer diameter. A shape of the first core portion 21 is not limited to the illustrated shape. For example, the distal portion 21a of the first core portion 21 may be formed in a flat plate shape. The first core portion 21 may also be formed to have a constant outer diameter from the distal side to the proximal side. In addition, for example, the core portion 20 can be a single continuous member instead of a plurality of members such as the first core portion 21 and the second core portion 22. The length of the first core portion 21 along the axial direction is not particularly limited. However, the length can be 20 to 1,000 mm.

A material of the first core portion 21 is not particularly limited. However, for example, Ni—Ti alloy, stainless steel, or superelastic alloy can be used. In addition, the material of the second core portion 22 is not particularly limited as long as the material of the second core portion 22 is different from the material of the first core portion 21. However, for example, stainless steel or cobalt-based alloy can be used. The first core portion 21 and the second core portion 22 can be joined to each other by welding, for example.

(Coil Portion)

The coil portion 30 is disposed so as to cover the first core portion 21 in a prescribed range over the axial direction. The coil portion 30 includes a wire rod spirally wound around the core portion 20 (first core portion 21) along the circumferential direction of the core portion 20.

The coil portion 30 according to the present embodiment is formed so as to be in close contact with an outer surface 20s of the core portion 20. However, the configuration is not limited thereto. For example, the coil portion 30 may be formed to be spaced from the outer surface 20s of the core portion 20. In addition, the coil portion 30 according to the present embodiment is formed so that a gap does not exist between the spirally wound turns of the coil portion 30 in a state where an external force is not applied. However, the configuration is not limited thereto. For example, in the state where the external force is not applied, the coil portion 30 may have the gap between the spirally wound turns.

The material of the coil portion 30 is not particularly limited. However, it is preferable that the coil portion 30 includes a material having radiopacity (X-ray contrast). For example, the material having the radiopacity includes noble metal such as gold, platinum, and tungsten, or a metallic material such as an alloy containing the above-described materials (for example, platinum-iridium alloy). In a case where the coil portion 30 includes the radiopaque material, the guide wire 10 can be inserted into the living body while a position of the distal portion of the guide wire 10 is confirmed using X-ray fluoroscopy.

The distal portion of the coil portion 30 is fixed to the vicinity of the distal portion of the first core portion 21 via a fixing material 31. The proximal portion of the coil portion 30 is fixed to the vicinity of the tapered portion 21b of the first core portion 21 via a fixing material 32. For example, the fixing materials 31 and 32 can include various adhesives or solder.

(Distal Side Coating Layer)

The distal side coating layer 40 includes a resin material, and is formed so as to cover the distal portion of the core portion 20 including the coil portion 30. It is preferable that the distal portion of the distal side coating layer 40 has a rounded shape as illustrated in FIG. 1 so as not to damage an inner wall of the body lumen. In addition, the proximal portion of the distal side coating layer 40 is located in the constant outer diameter portion 21c of the core portion 20 (first core portion 21).

It is preferable that the distal side coating layer 40 includes a material which can reduce friction. In this manner, friction resistance (sliding resistance) is reduced against the catheter into which the guide wire 10 is inserted or against the body lumen, thereby improving sliding performance. Therefore, operability of the guide wire 10 can be improved. In addition, since the sliding resistance of the guide wire 10 is reduced, the guide wire 10 can be more reliably prevented from being kinked (bent) or twisted.

It is preferable that the resin material configuring the distal side coating layer 40 is a relatively high flexible material. For example, the resin material can include polyolefin such as polyethylene and polypropylene, polyvinyl chloride, polyester (PET or PBT), polyamide, polyimide, polyurethane, polystyrene, polycarbonate, silicone resin, fluororesin (PTFE, ETFE, or PFA), or a composite material thereof, and various rubber materials such as latex rubber and silicone rubber, or a composite material containing two or more of the materials in combination. Among the above-described materials, from a viewpoint of further improving the flexibility, it is more preferable to use a urethane-based resin. In this manner, the distal portion of the guide wire 10 can be formed to be flexible. Accordingly, when the guide wire 10 is inserted into the body lumen, it is possible to prevent damage to the inner wall of the body lumen.

A thickness of the distal side coating layer 40 is not particularly limited. However, it is preferable that the thickness is 5 to 500 μm, for example. The distal side coating layer 40 is not limited to a single layer structure, and may be configured so that a plurality of layers are stacked one on another.

(Tubular Member)

The tubular member 50 is formed of a cylindrical (ring-shaped) member. The core portion 20 is inserted into the lumen of the tubular member 50. The tubular member 50 is formed in a state where the diameter of the lumen is reduced by cold forging (to be described later), and at least a portion of the inner surface 50s forming the lumen has a compressive bonding surface 70s which is compressively bonded to the outer surface 20s of the core portion 20 (constant outer diameter portion 21c of the first core portion 21).

Here, the term of “compressive bonding” means that two members are attached and fixed to each other in a state where a pressing force is applied in a direction in which both of these move close to each other. In the present embodiment, the diameter of the lumen of the tubular member 50 is reduced, thereby forming the compressive bonding surface 70s where the inner surface 50s of the tubular member 50 and the outer surface 20s of the core portion 20 are in contact with each other. In this manner, both of these are fixed to each other on the compressive bonding surface 70s in a state where the pressing force is applied in the direction in which both of these move close to each other.

In the present embodiment, the compressive bonding surface 70s is formed over the axial direction and the circumferential direction of the inner surface 50s of the tubular member 50. That is, the inner surface 50s of the tubular member 50 is formed in a state where the inner surface 50s is in close contact with the outer surface 20s of the core portion 20 without any substantial gap. In this manner, an area of the compressive bonding surface 70s increases. Accordingly, the tubular member 50 can be more firmly fixed to the core portion 20. Therefore, it is possible to further prevent the tubular member 50 from pulling away from the core portion 20.

As illustrated in FIG. 2, the distal portion 51 of the tubular member 50 is disposed so as to be in contact with at least a portion of the proximal end 41 of the distal side coating layer 40. In this manner, even if the guide wire 10 is bent when the guide wire 10 passes through the inside of the curved or meandering body lumen, the tubular member 50 restrains the proximal end 41 of the distal side coating layer 40 from being deformed. Accordingly, the distal side coating layer 40 can be prevented from being separated and stripped off from the core portion 20.

The distal portion 51 of the tubular member 50 is formed so that an outer diameter d11 is substantially constant along the axial direction. The outer diameter d11 of the distal portion 51 of the tubular member 50 is formed to be substantially the same as an outer diameter d2 of the proximal end 41 of the distal side coating layer 40. In addition, the outer surface 51s of the tubular member 50 includes a surface continuous with the outer surface 40s of the distal side coating layer 40. In this manner, the proximal end 41 of the distal side coating layer 40 can be prevented from being stripped off by getting caught on the inner wall of the body lumen or the catheter.

The outer diameter d11 of the distal portion 51 of the tubular member 50 may be formed to be larger than the outer diameter d2 of the proximal end 41 of the distal side coating layer 40. In this case, the proximal end 41 of the distal side coating layer 40 is located on the core portion 20 side (inward in the radial direction) further from the distal portion 51 of the tubular member 50. Accordingly, even if the proximal portion of the distal side coating layer 40 is stripped off, the proximal end 41 of the distal side coating layer 40 can be restrained from getting caught on the inner wall of the body lumen or the catheter. In addition, the outer diameter d11 of the distal portion 51 of the tubular member 50 may be formed to be smaller than the outer diameter d22 of the proximal portion (distal side further from the proximal end 41) of the distal side coating layer 40.

In the description herein, the term of “continuous surface” means a smooth surface formed to such an extent that the guide wire 10 does not get caught on the inner wall of the body lumen or the catheter. The continuous surface is not limited to a flat surface (refer to FIG. 2) formed substantially parallel to the axial direction. The continuous surface can also include a curved surface or an inclined surface in the axial direction.

The proximal portion 52 of the tubular member 50 has a tapered shape whose outer diameter d12 gradually decreases (inclined in the axial direction) toward the proximal side, starting from the proximal end of the distal portion 51. The proximal portion 52 of the tubular member 50 has the tapered shape. Accordingly, a step difference (portion whose outer diameter is rapidly changed) can be eliminated between the tubular member 50 and the core portion 20. In this manner, the portion having the step difference can be prevented from getting caught on the inner wall of the body lumen or the catheter. Furthermore, rigidity (flexural rigidity or torsional rigidity) of the guide wire 10 can be gradually changed. Accordingly, it is possible to restrain a sudden change in the rigidity. As a result, followability of the guide wire 10 following blood vessels can be improved, and the guide wire 10 can be prevented from being bent.

In the present embodiment, an inclination angle in the axial direction of the tapered shape of the proximal portion 52 of the tubular member 50 is formed to be substantially constant along the axial direction. In this manner, the rigidity along the axial direction of the guide wire 10 can be more smoothly changed. The inclination angle of the tapered shape of the proximal portion 52 of the tubular member 50 may be changed along the axial direction. For example, a portion having a relatively large inclination angle and a portion having a relatively small inclination angle (including a case where the inclination angle is zero) may be alternately and repeatedly formed multiple times. In addition, the proximal portion 52 may be formed in a stepwise shape where the outer diameter d12 gradually decreases. In addition, the outer diameter d12 of the proximal portion 52 of the tubular member 50 may be formed to be substantially constant along the axial direction.

The tubular member 50 includes a material harder than the resin material configuring the distal side coating layer 40. As the material, it is preferable to use a metallic material. For example, the metallic material can include stainless steel (SUS), superelastic alloy, cobalt-based alloy, noble metal such as gold, platinum, and tungsten, or an alloy containing the above-described materials (for example, platinum-iridium alloy). Among the above-described materials, it is preferable to use relatively inexpensive stainless steel (SUS).

The length of the tubular member 50 along the axial direction is not particularly limited. However, for example, the length can be 0.5 to 2 mm.

(Hydrophilic Coating Layer)

It is preferable that the distal side coating layer 40 and the tubular member 50 are covered with a hydrophilic coating layer (not illustrated). When covered with the hydrophilic coating layer, sliding performance is improved. Accordingly, the guide wire 10 can be further prevented from getting caught on the inner wall of the body lumen or the catheter.

The material of the hydrophilic coating layer is not particularly limited. However, for example, the material can include known hydrophilic materials formed of cellulose-based polymer materials, polyethylene oxide-based polymer materials, maleic anhydride-based polymer materials (for example, maleic anhydride copolymer such as methyl vinyl ether-maleic anhydride copolymer), acrylamide-based polymer materials (for example, polyacrylamide, block copolymer of polyglycidyl methacrylate-dimethyl acrylamide (PGMA-DMAA)), water-soluble nylon, polyvinyl alcohol, and polyvinyl pyrrolidone.

The thickness of the hydrophilic coating layer is not particularly limited. However, for example, it is preferable that the thickness is 0.1 to 100 μm.

(Proximal Side Coating Layer)

As illustrated in FIG. 1, the proximal side coating layer 60 is formed so as to cover the proximal portion of the core portion 20 (second core portion 22). The proximal side coating layer 60 has an inner layer 61 for covering the outer surface 20s of the core portion 20, an outer layer 62 for covering the outer surface of the inner layer 61, and a linear body 63 which is spirally wound around the outer surface of the outer layer 62.

The linear body 63 is spirally wound so that the turns adjacent to each other along the outer surface of the outer layer 62 are spaced apart from each other. In this manner, the outer surface of the outer layer 62 has irregularities.

The material of the inner layer 61, the outer layer 62, and the linear body 63 is not particularly limited. However, for example, fluorine-based resins such as PTFE and ETFE can be used.

The proximal side coating layer 60 is not limited to the above-described configuration, and may be a single layer, for example.

A manufacturing method of the guide wire 10 according to the first embodiment will be described.

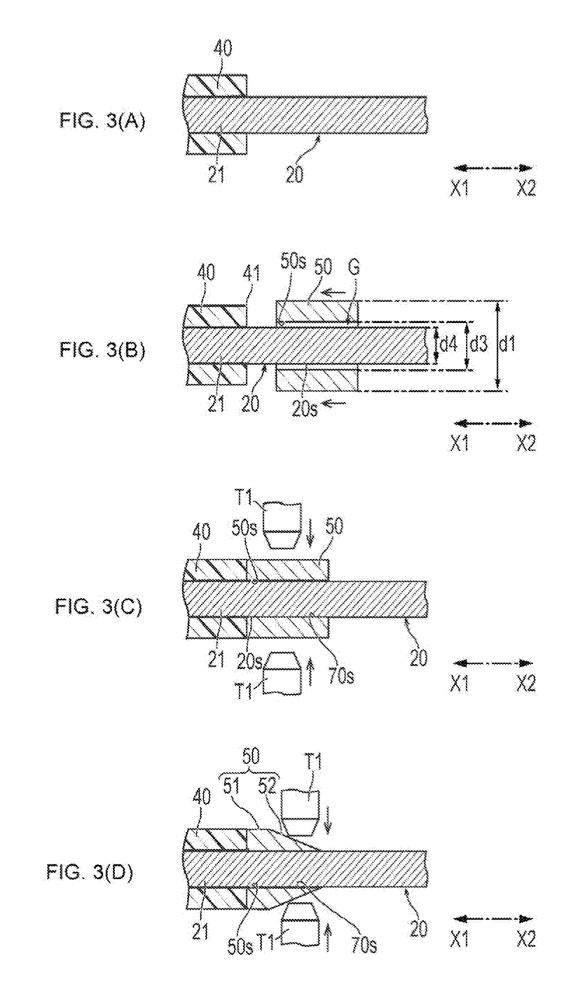

FIGS. 3(A) to 3(D) are views schematically illustrating the manufacturing method of the guide wire 10, and FIG. 4 is a schematic view for describing swaging (cold forging).

First, as illustrated in FIG. 3(A), the first core portion 21 having the coil portion 30 (not illustrated) and the distal side coating layer 40 formed therein is prepared.

Next, as illustrated in FIG. 3(B), the first core portion 21 is inserted into the lumen of the tubular member 50, and the distal end of the tubular member 50 is attached to the proximal end 41 of the distal side coating layer 40. At this stage, an inner diameter (diameter of the lumen) d3 of the tubular member 50 is larger than an outer diameter d4 of the first core portion 21. In this manner, a gap G is formed between the inner surface 50s of the tubular member 50 and the outer surface 20s of the first core portion 21. Accordingly, the first core portion 21 can be easily inserted into the lumen of the tubular member 50. In addition, the outer diameter d1 of the tubular member 50 is substantially constant along the axial direction.

Next, as illustrated in FIG. 3(C), swaging (cold forging) is performed on the tubular member 50 so as to reduce the diameter of the lumen of the tubular member 50 (cold forging step). In this manner, the inner surface 50s of the tubular member 50 forms the compressive bonding surface 70s which is compressively bonded to the outer surface 20s of the first core portion 21. The tubular member 50 is firmly fixed to the first core portion 21 by the compressive bonding surface 70s. In addition, as illustrated in FIG. 2, the outer diameter d11 of the distal portion 51 of the tubular member 50 and the outer diameter d2 of the proximal end 41 of the distal side coating layer 40 are substantially the same as each other. The outer surface 51s of the distal portion 51 of the tubular member 50 is processed so as to include a smooth surface continuous with the outer surface 40s of the distal side coating layer 40. As will be described later, according to the swaging, the outer diameter d1 of the tubular member 50 can be very accurately adjusted. Therefore, it is not necessary to carry out post-processing work such as grinding the outer surface 51s of the tubular member 50 in order to form the smooth surface continuous with the outer surface 40s of the distal side coating layer 40.

Next, the swaging is further performed. As illustrated in FIG. 3(D), a tapered shape whose outer diameter gradually decreases toward the proximal side is formed in the proximal portion 52 of the tubular member 50 (cold forging step). Concurrently with the processing for reducing the diameter of the lumen of the tubular member 50 illustrated in FIG. 3(C), the proximal portion 52 may be formed into a tapered shape.

Here, the term of “swaging” represents a processing method in which a metallic target member is interposed between a plurality of dies T1 and the target member is subjected to compression molding between one die T1 and the other die T1. In the present embodiment, the term means that the diameter of the lumen of the tubular member 50 is reduced by cold forging in which a compressive force (striking force) facing toward the core portion 20 side (inward in the radial direction) is applied to the tubular member 50 multiple times.

In the swaging according to the present embodiment, as illustrated in FIG. 4, the plurality of dies T1 are first installed so as to surround the tubular member 50 in the circumferential direction. Next, while the die T1 is rotated in the circumferential direction, the die T1 is caused to repeatedly reciprocate inward and outward in the radial direction (direction of an arrow R in FIG. 4). The compressive force facing toward the core portion 20 side (inward in the radial direction) is applied to the tubular member 50 multiple times. In this manner, the tubular member 50 is compressed inward in the radial direction so as to gradually reduce the diameter of the lumen of the tubular member 50. According to the swaging, the inner diameter d3 of the tubular member 50 and the outer diameter d1 (refer to FIG. 3(B)) can be processed to a desired size with high processing accuracy by adjusting the number of times to apply the compressive force to the tubular member 50 or a magnitude of the compressive force.

Finally, the guide wire 10 is obtained by joining the first core portion 21 to the second core portion 22 having the proximal side coating layer 60 formed therein.

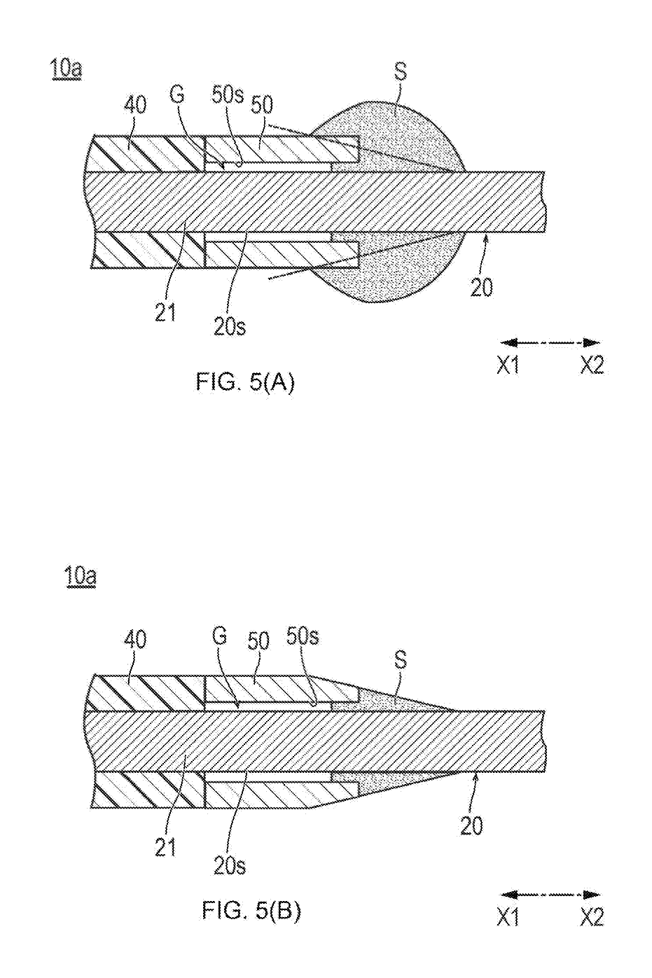

Next, referring to FIGS. 5 to 7, an operation of the guide wire 10 and the manufacturing method of the guide wire 10 according to the present embodiment will be described.

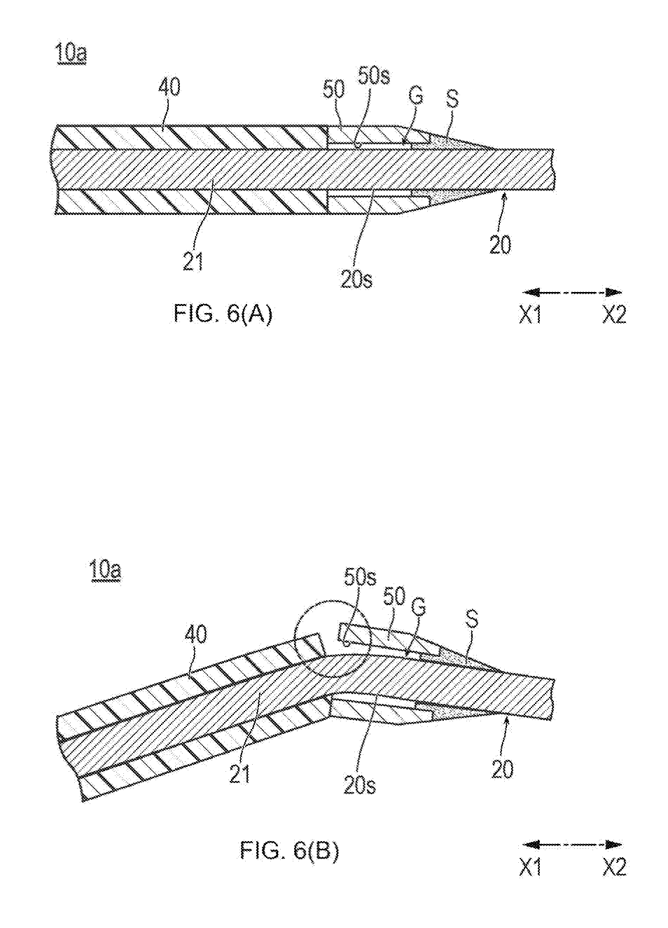

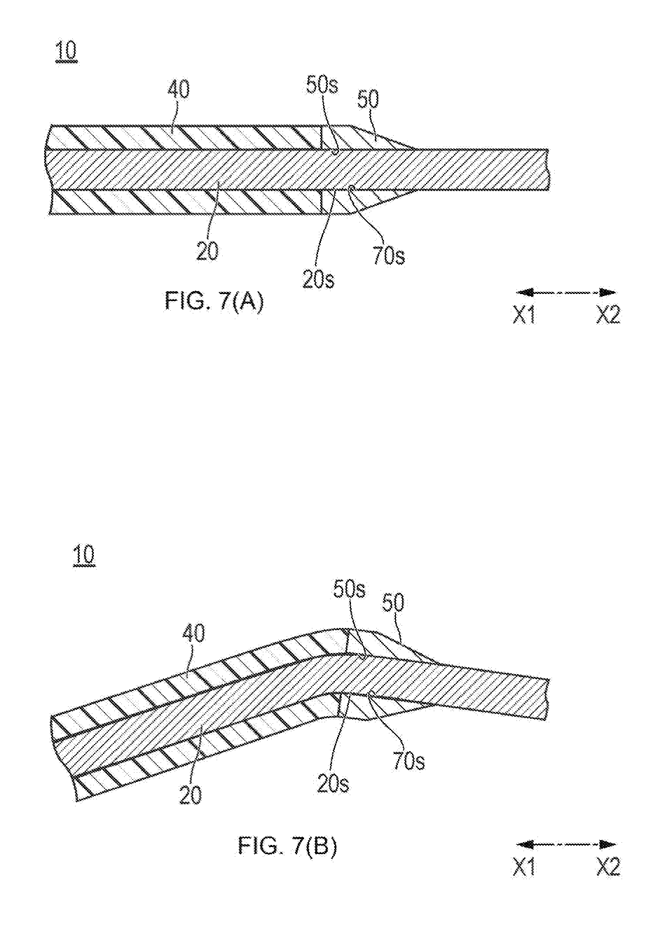

FIGS. 5 (A) and 5(B) are views schematically illustrating a manufacturing method of a guide wire 10a according to a comparative example. FIG. 6(A) an axial cross-sectional view illustrating the guide wire 10a, and FIG. 6 (B) is an axial cross-sectional view illustrating a state where the guide wire 10a is bent. In addition, FIG. 7(A) is an axial cross-sectional view illustrating the guide wire 10 according to the present embodiment, and FIG. 7(B) is an axial cross-sectional view illustrating a state where the guide wire 10 is bent.

In the manufacturing method of the guide wire 10a according to the comparative example, the first core portion 21 is disposed by being inserted into the lumen of the tubular member 50 (refer to FIG. 3(B)). Thereafter, swaging is not performed, and the tubular member 50 is fixed to the first core portion 21 by using solder S. As illustrated in FIG. 5(A), the solder S is melted and disposed so as to fill a step difference between the first core portion 21 and the proximal end of the tubular member 50, and then, the solder S is hardened.

Next, as illustrated in FIG. 5(B), a superfluous portion of the proximal portion of the tubular member 50 and the solder S is removed by grinding (refer to a broken line in FIG. 5(A)). The proximal portion of the tubular member 50 and the solder S are formed in a tapered shape whose outer diameter gradually decreases toward the proximal side.

As described above, according to the manufacturing method of the guide wire 10a in the comparative example, the proximal portion of the tubular member 50 and the portion of the solder S are removed by grinding. Consequently, the proximal portion of the tubular member 50 becomes brittle, thereby causing a possibility of damage. In addition, there is a possibility that the physical properties of the core portion 20 and the distal side coating layer 40 may deteriorate due to the influence of heat when the solder S is melted. The quality of the guide wire 10a is thus degraded in the manufacturing method of the comparative example.

In addition, in order to carry out the grinding work for the tubular member 50, the material of the tubular member 50 is limited to materials which are relatively easy to be processed, for example, such as platinum-iridium alloy. Furthermore, when the proximal portion of the tubular member 50 and the portion of the solder S are removed, dust such as abrasive powder is generated. Consequently, it becomes necessary to carry out work to remove the dust. In addition, the solder S is used. Consequently, material cost (number of components) increases compared to the manufacturing method of the guide wire 10 according to the present embodiment. Furthermore, when the outer surface of the solder S is covered with a hydrophilic resin, it becomes necessary to perform a surface treatment step for improving the wettability of the outer surface of the solder S. In addition, the effects of the material of the solder S on the living body must be considered when choosing which solder to use. The work for manufacturing the guide wire 10 is thus complicated, and the manufacturing cost increases, in the manufacturing method of the comparative example.

In addition, as illustrated in FIG. 6(A), the guide wire 10a manufactured by the manufacturing method according to the comparative example has a gap G formed between the inner surface 50s of the tubular member 50 and the outer surface 20s of the core portion 20. Therefore, when the guide wire 10a passes through the inside of the curved or meandering body lumen, the guide wire 10a may be bent and the gap G may be widened as illustrated by a portion surrounded with a broken line in FIG. 6(B), in some cases. In this case, an end portion of the tubular member 50 protrudes outward in the radial direction, thereby making it possible for the inner wall inside the body lumen to become damaged, or the tubular member 50 to get caught on the catheter.

In order to solve the above-described problem, in the manufacturing method of the guide wire 10 according to the present embodiment, the diameter of the lumen of the tubular member 50 is reduced by swaging, and the tubular member 50 is fixed to the core portion 20. Accordingly, as illustrated in FIG. 7(A), the compressive bonding surface 70s which is compressively bonded to the outer surface 20s of the core portion 20 can be formed in at least a portion of the inner surface 50s of the tubular member 50. Therefore, even if the guide wire 10 is bent as illustrated in FIG. 7(B), while the tubular member 50 maintains a state of being in close contact with the core portion 20 on the compressive bonding surface 70s, the tubular member 50 is deformed to follow the deformation of the core portion 20. The compressive bonding surface 70s is formed in this way, thereby allowing the tubular member 50 to be firmly fixed to the core portion 20. Therefore, the tubular member 50 can be restrained from pulling away from the core portion 20. In this manner, the tubular member 50 can be prevented from slipping out of the core portion 20, and the tubular member 50 can be restrained from damaging the inner wall inside the body lumen or getting caught on the catheter.

In addition, since swaging is used, a fixing member such as solder S for fixing the tubular member 50 to the core portion 20 does not need to be used. Accordingly, the number of components can be reduced. Furthermore, since the fixing member is not used, it is not necessary to perform a test for confirming the influence of the fixing member on the living body, or a surface treatment step for improving the wettability. In addition, grinding work to remove a portion of the proximal portion of the tubular member 50 is not needed. Accordingly, a relatively inexpensive material such as stainless steel (SUS) can be used as the material of the tubular member 50. In this manner, the work for manufacturing the guide wire 10 can be further simplified, and the manufacturing cost including the material cost can be reduced. In addition, the tubular member 50 subjected to the swaging (cold forging) has improved strength through hardening work, making it less likely for the tubular member 50 to get damaged.

As described above, the guide wire 10 according to the present embodiment includes the elongated core portion 20, the distal side coating layer (resin coating layer) 40 that is the resin material, and that covers the distal portion of the core portion 20, and the metallic tubular member 50 that has the lumen into which the core portion 20 is inserted, and that is disposed so as to be in contact with at least the portion of the proximal end 41 of the distal side coating layer 40. The tubular member 50 is formed in a state where the diameter of the lumen is reduced by cold forging, and at least a portion of the inner surface 50s forming the lumen has the compressive bonding surface 70s which is compressively bonded to the outer surface 20s of the core portion 20.

The above-described guide wire 10 includes the tubular member 50 disposed so as to be in contact with at least the portion of the proximal end 41 of the distal side coating layer 40. Accordingly, the distal side coating layer 40 can be prevented from being separated and stripped off from the core portion 20. In addition, the tubular member 50 includes the compressive bonding surface 70s. Accordingly, the tubular member 50 can be restrained from pulling away from the core portion 20. In this manner, the tubular member 50 can be prevented from slipping out of the core portion 20, and the tubular member 50 can be prevented from damaging the inner wall inside the body lumen or getting caught on the catheter.

In addition, the compressive bonding surface 70s is formed over the entire inner surface 50s of the tubular member 50. In this manner, the inner surface 50s of the tubular member 50 is formed in a state where the inner surface 50s is in close contact with the outer surface 20s of the core portion 20 without any substantial gap. An area of the compressive bonding surface 70s increases. Accordingly, the tubular member 50 can be more firmly fixed to the core portion 20. Therefore, the tubular member 50 can be further restrained from pulling away from the core portion 20. In this manner, the tubular member 50 can be prevented from slipping out of the core portion 20, and the tubular member 50 can be prevented from damaging the inner wall inside the body lumen or getting caught on the catheter.

In addition, the outer surface 51s of the distal portion 51 of the tubular member 50 includes the surface continuous with the outer surface 40s of the distal side coating layer 40. Accordingly, when the guide wire 10 is inserted into the body lumen or into the lumen of the catheter, the guide wire 10 can be prevented from getting caught on the inner wall inside the body lumen or the catheter.

In addition, the proximal portion 52 of the tubular member 50 has the tapered shape whose outer diameter d12 gradually decreases toward the proximal side. In this manner, the step difference can be eliminated between the proximal portion 52 of the tubular member 50 and the core portion 20. Accordingly, the proximal portion 52 of the tubular member 50 can be effectively prevented from getting caught on the inner wall of the body lumen or the catheter.

The manufacturing method of the guide wire 10 according to the present embodiment has the step of inserting the core portion 20 into the lumen of the tubular member 50, and disposing the tubular member 50 so as to be in contact with at least the portion of the proximal end 41 of the distal side coating layer 40, and the cold forging step of reducing the diameter of the lumen of the tubular member 50 by cold forging, and forming the compressive bonding surface 70s compressively bonded to the outer surface 20s of the core portion 20 in at least the portion of the inner surface 50s forming the lumen.

According to the manufacturing method of the above-described guide wire 10, the tubular member 50 is disposed so as to be in contact with at least the portion of the proximal end 41 of the distal side coating layer 40. Accordingly, the distal side coating layer 40 can be prevented from being separated and stripped off from the core portion 20. In addition, the diameter of the lumen of the tubular member 50 is reduced by the cold forging, and the compressive bonding surface 70s which is compressively bonded to the inner surface 50s of the tubular member 50 is formed on the outer surface 20s of the core portion 20. Accordingly, the tubular member 50 can be restrained from pulling away from the core portion 20. In this manner, the tubular member 50 can be prevented from slipping out of the core portion 20, and the tubular member 50 can be prevented from damaging the inner wall inside the body lumen or getting caught on the catheter. In addition, since the cold forging is performed, the tubular member 50 can be processed and hardened to improve the strength, making it less likely for the tubular member 50 to get damaged.

In addition, in the cold forging step, the compressive force facing the core portion 20 side is applied to the tubular member 50, and the tubular member 50 is compressed against the core portion 20 side, thereby performing the swaging for reducing the diameter of the lumen of the tubular member 50. The compressive force applied to the tubular member 50 is adjusted. Accordingly, the tubular member 50 can be subjected to compression molding with high processing accuracy. Furthermore, since the swaging is performed, the tubular member 50 can be fixed to the core portion 20 without using the fixing material such as the solder S. In this manner, the work for manufacturing guide wire 10 can be simplified, and the manufacturing cost can be reduced.

In addition, in the cold forging step, the compressive bonding surface 70s is formed on the entire inner surface 50s of the tubular member 50. Accordingly, the inner surface 50s of the tubular member 50 is formed in a state where the inner surface 50s is in close contact with the outer surface 20s of the core portion 20 without any substantial gap. According to the cold forging (swaging), the diameter of the lumen of the tubular member 50 can be reduced. Accordingly, the compressive bonding surface 70s can be relatively easily formed over the entire inner surface 50s of the tubular member 50. In this manner, the area of the compressive bonding surface 70s increases. Accordingly, the tubular member 50 can be more firmly fixed to the core portion 20. Therefore, the tubular member 50 can be further prevented from pulling away from the core portion 20.

In addition, in the cold forging step, the outer surface 51s of the distal portion 51 of the tubular member 50 is formed on the surface continuous with the outer surface 40s of the distal side coating layer 40. According to the cold forging (swaging), the outer diameter of the tubular member 50 can be very accurately adjusted. Therefore, it is not necessary to carry out post-processing work such as grinding the outer surface 51s of the tubular member 50 in order to form the smooth surface continuous with the outer surface 40s of the distal side coating layer 40.

### Second Embodiment

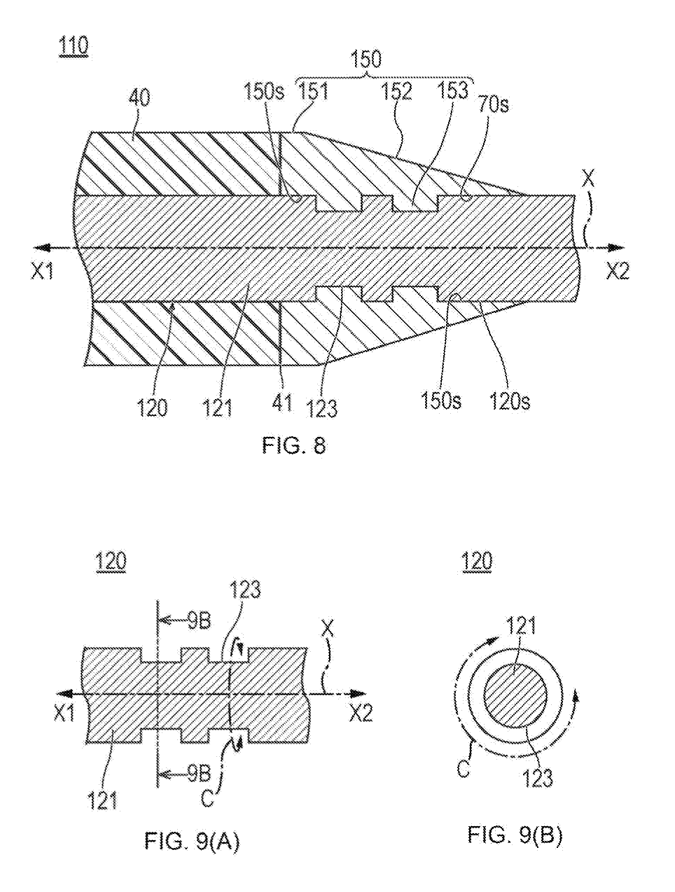

Next, referring to FIGS. 8 to 10, a second embodiment will be described. The same reference numerals will be given to configurations the same as those according to the above-described first embodiment, and description thereof will be omitted. In addition, points particularly not described in the second embodiment can be configured similarly to the above-described first embodiment.

FIG. 8 is an axial cross-sectional view illustrating an enlarged main portion of a guide wire 110 according to the second embodiment. FIG. 9(A) is an axial cross-sectional view of a core portion 120 according to the second embodiment, and FIG. 9(B) is a cross-sectional view taken along line 9B-9B illustrated in FIG. 9(A).

In the guide wire 110 according to the second embodiment, a first core portion 121 (core portion 120) has at least one concave portion (corresponding to an engagement portion) 123 which engages with the tubular member 150. The tubular member 150 has at least one convex portion 153 (corresponding to an engagement pairing portion) which corresponds to and engages with the concave portion 123. The above-described point is different from that according to the above-described first embodiment.

The concave portion 123 of the first core portion 121 is disposed in at least a portion of an outer surface 120s of the first core portion 121, and has a concave shape recessed inward in the radial direction.

Similarly to the above-described first embodiment, the tubular member 150 has a distal portion 151 whose outer diameter is formed to be substantially constant along the axial direction, and a proximal portion 152 having a tapered shape whose outer diameter gradually decreases toward the proximal side.

The convex portion 153 of the tubular member 150 is disposed in at least a portion of an inner surface 150s of the tubular member 150, and has a shape which protrudes in a convex shape toward the first core portion 121 side (inward in the radial direction). The convex portion 153 of the tubular member 150 has a shape into which the concave portion 123 of the first core portion 121 is transferred. That is, the convex portion 153 of the tubular member 150 has a fitting shape fittable to the entire concave portion 123.

As illustrated in FIGS. 9(A) and 9(B), the concave portion 123 of the first core portion 121 is formed in a band shape along the entire circumferential direction.

A manufacturing method of the guide wire 110 according to the second embodiment will be described.

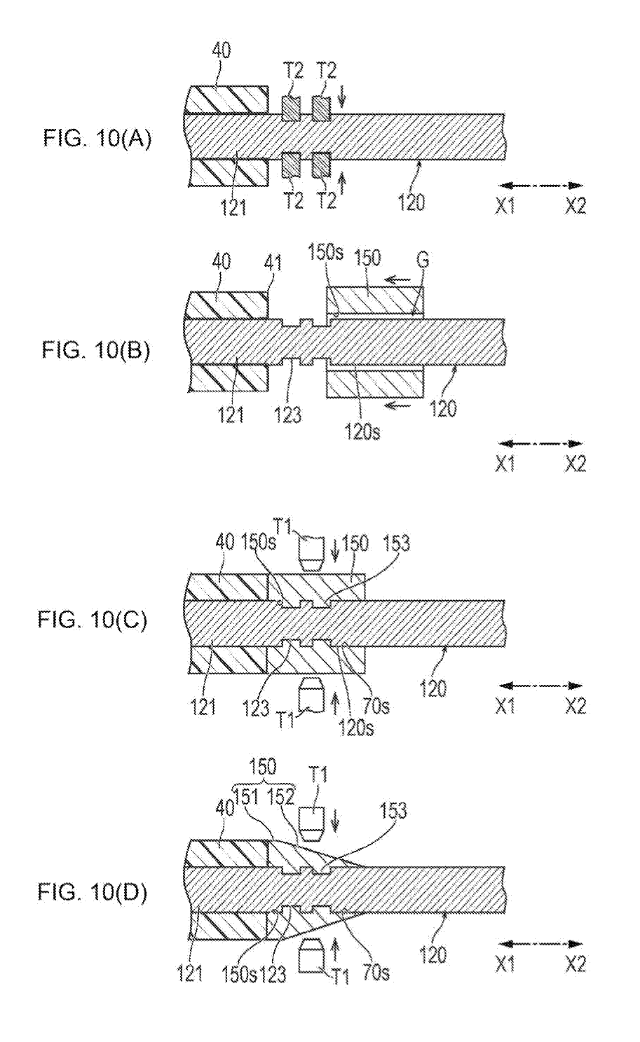

FIGS. 10(A) to 10(D) are views schematically illustrating the manufacturing method of the guide wire 110 according to the second embodiment.

First, as illustrated in FIG. 10(A), the first core portion 121 having the coil portion 30 (not illustrated) and the distal side coating layer 40 formed therein is prepared, and through caulking using a tool T2, the concave portion 123 is formed in a portion of the first core portion 121.

Next, similarly to the above-described first embodiment, as illustrated in FIG. 10(B), the first core portion 121 is inserted into the lumen of the tubular member 150, and the distal end of the tubular member 150 is attached to the proximal end 41 of the distal side coating layer 40. The outer diameter of the tubular member 150 is substantially constant along the axial direction, and the gap G is formed between the inner surface 150s of the tubular member 150 and the outer surface 120s of the first core portion 121. In addition, at this stage, the inner diameter (diameter of the lumen) of the tubular member 150 is substantially constant along the axial direction, and the convex portion 153 is not formed on the inner surface 150s.

Next, as illustrated in FIG. 10(C), the tubular member 150 is subjected to the swaging (cold forging), and the diameter of the lumen of the tubular member 150 is reduced. In this manner, the inner surface 150s of the tubular member 150 comes into contact with the outer surface 120s of the first core portion 121. Furthermore, if the swaging is performed, a portion of the tubular member 150 enters the concave portion 123 of the first core portion 121, and the convex portion 153 into which the shape of the concave portion 123 of the first core portion 121 is transferred is formed on the inner surface 150s of the tubular member 150. In this manner, the compressive bonding surface 70s which is compressively bonded to the outer surface 120s of the first core portion 121 is formed on the inner surface 150s of the tubular member 150, and the convex portion 153 of the tubular member 150 which engages with the concave portion 123 of the first core portion 121 is formed.

According to the swaging, the number of times to apply the compressive force to the tubular member 150 and the magnitude of the compressive force are adjusted. Accordingly, a portion of the tubular member 150 is caused to enter the concave portion 123 of the first core portion 121. In this manner, it is possible to relatively easily perform a process for forming the convex portion 153. In the present embodiment, the portion of the tubular member 150 is caused to enter the entire concave portion 123 of the first core portion 121, thereby forming the convex portion 153 having the fitting shape fittable to the concave portion 123.

Thereafter, similarly to the above-described first embodiment, the swaging is further performed. As illustrated in FIG. 10(D), a tapered shape whose outer diameter gradually decreases toward the proximal side is formed in the proximal portion 152 of the tubular member 150. Concurrently with the processing for reducing the diameter of the lumen of the tubular member 150, the tapered shape of the proximal portion 152 may be formed.

Finally, the guide wire 110 is obtained by joining the first core portion 121 to the second core portion 22 having the proximal side coating layer 60 formed therein.

The guide wire 110 and the manufacturing method of the guide wire 110 according to the second embodiment can also achieve an advantageous effect the same as that according to the above-described first embodiment.

In addition, the core portion 120 according to the present embodiment has at least one concave portion (engagement portion) 123 which engages with the tubular member 150, and the tubular member 150 has at least one convex portion (engagement pairing portion) 153 which corresponds to and engages with the concave portion 123. In this manner, the concave portion (engagement portion) 123 engages with the convex portion (engagement pairing portion) 153. Accordingly, the tubular member 150 can be more reliably prevented from slipping out of the core portion 120.

In addition, the concave portion (engagement portion) 123 is disposed in at least the portion of the outer surface 120s of the core portion 120, and has a concave shape recessed inward in the radial direction. The convex portion (engagement pairing portion) 153 is disposed in at least the portion of the inner surface 150s of the tubular member 150, and has a convex shape protruding to the core portion 120 side. The concave portion 123 of the core portion 120 engages with the convex portion 153 of the tubular member 150. Accordingly, the tubular member 150 can be restrained from moving relative to the core portion 120. As a result, the tubular member 150 can be more reliably prevented from slipping out of the core portion 120. Furthermore, as in the present embodiment, in a case where the convex portion 153 has the fitting shape fittable to the concave portion 123, the tubular member 150 can be more reliably prevented from moving relative to the core portion 120 in the axial direction or in the circumferential direction.

The manufacturing method of the guide wire 110 according to the present embodiment further has a step of forming at least one concave portion (engagement portion) 123 capable of engaging with the tubular member 50 in the core portion 120 prior to the cold forging step. In the cold forging step, at least one convex portion (engagement pairing portion) 153 which corresponds to and engages with the concave portion 123 is formed. According to the cold forging, a portion of the tubular member 150 is caused to enter the concave portion 123 of the first core portion 121. In this manner, it is possible to relatively easily perform the process for forming the convex portion 153. Furthermore, as in the present embodiment, the portion of the tubular member 150 is caused to enter the entire concave portion 123 of the first core portion 121. Accordingly, it is possible to form the convex portion 153 having the fitting shape into which the shape of the concave portion 123 is transferred. In this manner, the tubular member 150 can be more reliably prevented from moving relative to the core portion 120 in the axial direction and the circumferential direction.

Next, modification examples according to the above-described second embodiment will be described. In the description of each modification example, the same reference numerals will be given to configurations the same as those according the above-described second embodiment, and description thereof will be omitted. In addition, points particularly not described in each modification example can be configured similarly to the above-described second embodiment.

In each modification example, a shape of the concave portion of the core portion is different from that according to the second embodiment. Although the convex portion of the tubular member is not specifically described, the convex portion has an engageable shape corresponding to the concave portion according to each modification example.

### Modification Example 1 of Second Embodiment

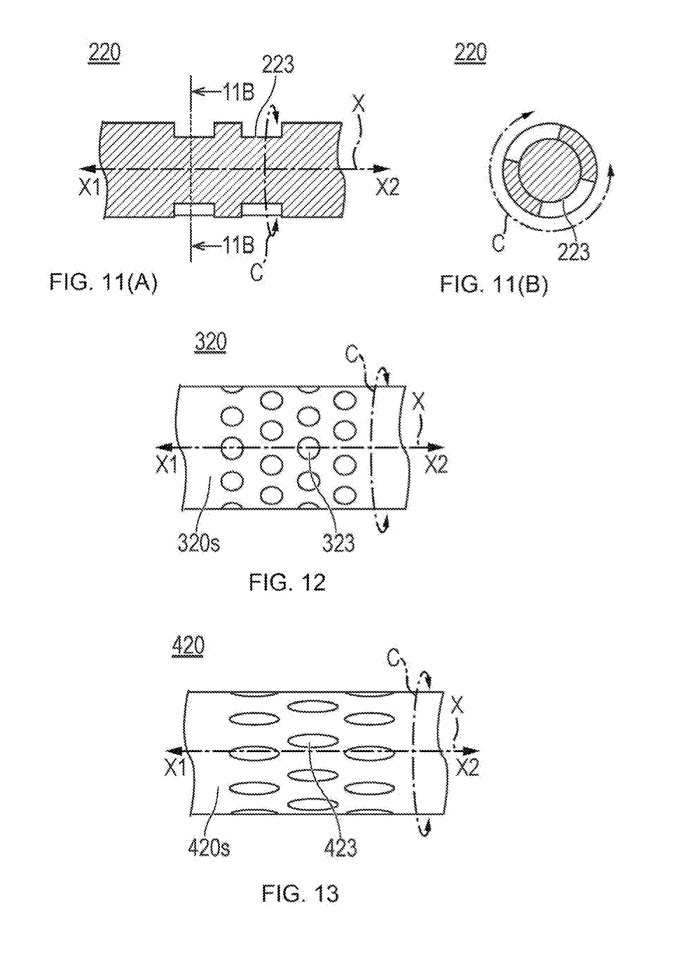

FIG. 11(A) is an axial cross-sectional view of a core portion 220 according to Modification Example 1 of the second embodiment, and FIG. 11(B) is a cross-sectional view taken along line 11B-11B illustrated in FIG. 11(A).

In the above-described second embodiment, the concave portion 123 is formed over the entire periphery of the core portion 220 in the circumferential direction. However, as illustrated in FIG. 11(B), unlike in the above-described second embodiment, in the core portion 220 according to Modification Example 1, a concave portion 223 is formed in a rectangular shape in a portion in the circumferential direction. The guide wire including the core portion 220 according to this modification example can also achieve an advantageous effect the same as that according to the above-described second embodiment.

### Modification Example 2 of Second Embodiment

FIG. 12 is a side view illustrating a core portion 320 according to Modification Example 2 of the second embodiment.

As illustrated in FIG. 12, unlike in the above-described second embodiment, the core portion 320 according to Modification Example 2 has a plurality of circular concave portions 323 which are respectively arranged in the circumferential direction and the axial direction of an outer surface 320s thereof. The guide wire having the core portion 320 according to this modification example can also achieve an advantageous effect the same as that according to the above-described second embodiment.

In addition, the plurality of concave portions 323 are disposed in the outer circumferential direction and the axial direction of the outer surface 320s of the core portion 320. In this manner, the tubular member can be prevented from moving relative to the core portion 320 in the axial direction and from slipping out of the core portion 120, and the tubular member can be prevented from moving (rotating) relative to the core portion 320 in the circumferential direction.

### Modification Example 3 of Second Embodiment

FIG. 13 is a side view illustrating a core portion 420 according to Modification Example 3 of the second embodiment.

As illustrated in FIG. 13, unlike in the above-described second embodiment, the core portion 420 according to Modification Example 3 has a plurality of elliptical concave portion 423 which are respectively arranged in the circumferential direction and the axial direction of an outer surface 420s thereof. The guide wire having the core portion 420 according to this modification example can also achieve an advantageous effect the same as that according to Modification Example 2 of the above-described second embodiment.

### Modification Example 4 of Second Embodiment

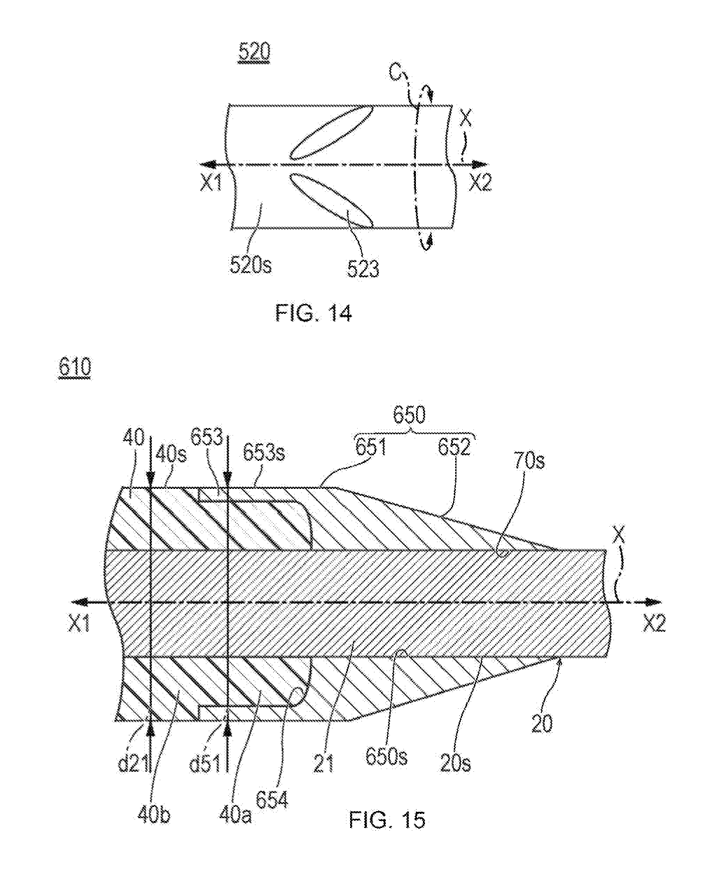

FIG. 14 is a side view illustrating a core portion 520 according to Modification Example 4 of the second embodiment.

As illustrated in FIG. 14, unlike in the above-described second embodiment, the core portion 520 according to Modification Example 4 has a plurality of elliptical concave portions 523 which are arranged in the circumferential direction of an outer surface 520s thereof. Furthermore, at least two adjacent concave portions 523 in the plurality of concave portions 523 are disposed so that the distance therebetween is far away from each other toward the proximal side. Elliptical long axes are inclined in directions respectively opposite to the axial direction. That is, the elliptical long axes of at least two adjacent concave portions 523 are disposed in a V-shape on the outer surface 520s of the core portion 520. The guide wire including the core portion 520 according to this modification example can also achieve an advantageous effect the same as that according to the above-described second embodiment.

In addition, according to the core portion 520 in the modification example, the elliptical long axes of at least two adjacent concave portions 523 arranged in the circumferential direction are formed in the V-shape on the outer surface 520s of the core portion 520. In this manner, the movement of the tubular member is restricted in the axial direction, and the tubular member can be further prevented from slipping out of the core portion.

### Third Embodiment

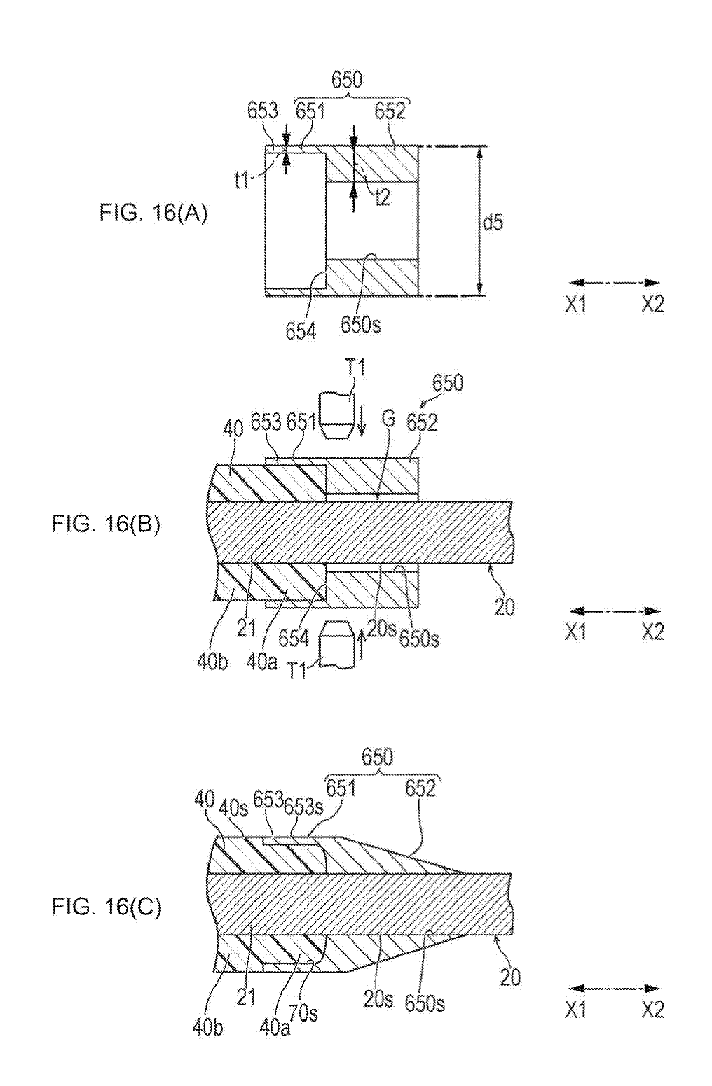

Next, referring to FIGS. 15 and 16, a third embodiment will be described. The same reference numerals will be given to configurations the same as those according to the above-described first embodiment, and description thereof will be omitted. In addition, points particularly not described in the third embodiment can be configured similarly to the above-described first embodiment.

FIG. 15 is an axial cross-sectional view illustrating an enlarged main portion of a guide wire 610 according to the third embodiment.

As illustrated in FIG. 15, unlike in the above-described first embodiment, in the guide wire 610 according to the third embodiment, a distal portion 651 of a tubular member 650 has an extending portion 653 which extends to the distal side so as to cover at least a portion of the proximal portion of the distal side coating layer 40. Hereinafter, a portion covered with the extending portion 653 in the proximal portion of the distal side coating layer 40 is referred to as a first proximal portion 40a, and a portion located on the distal side further from the extending portion 653 is referred to as a second proximal portion 40b.

An outer diameter d51 of the extending portion 653 of the tubular member 650 is formed to be substantially the same as an outer diameter d21 of the second proximal portion 40b of the distal side coating layer 40. That is, an outer surface 653s of the extending portion 653 of the tubular member 650 includes a surface continuous with the outer surface 40s of the second proximal portion 40b of the distal side coating layer 40.

A manufacturing method of the guide wire 610 according to the third embodiment will be described.

FIGS. 16(A) to 16(C) are views schematically illustrating the manufacturing method of the guide wire 610 according to the third embodiment.

First, as illustrated in FIG. 16(A), the tubular member 650 is prepared. A thickness t1 of the extending portion 653 of the tubular member 650 is thinner than a thickness t2 of a proximal portion 652. In addition, an outer diameter d5 of the tubular member 650, the thickness t1 of the extending portion 653, and the thickness t2 of the proximal portion 652 are substantially constant along the axial direction. Therefore, the inner surface 650s of the tubular member 650 has a step difference portion 654 formed in a portion switched from the proximal portion 652 to the extending portion 653.

Next, similarly to the above-described first embodiment, as illustrated in FIG. 16(B), the first core portion 21 is inserted into the lumen of the tubular member 650, and the step difference portion 654 of the tubular member 650 is attached to the distal side coating layer 40. In this manner, the extending portion 653 is disposed so as to cover the outer periphery of the first proximal portion 40a of the distal side coating layer 40.

Next, the swaging is performed on the tubular member 650 so as to reduce the diameter of the lumen of the tubular member 650. In this manner, the compressive bonding surface 70s which is compressively bonded to the outer surface 20s of the core portion 20 and the outer surface 40s of the first proximal portion 40a of the distal side coating layer 40 is formed on the inner surface 650s of the tubular member 650. In addition, through the swaging, the outer surface 653s of the extending portion 653 of the tubular member 650 is processed so as to include a surface continuous with the outer surface 40s of the second proximal portion 40b of the distal side coating layer 40.

According to the swaging, the outer diameter of the extending portion 653 of the tubular member 650 can be relatively easily and very accurately adjusted. Therefore, it is not necessary to carry out post-processing work such as grinding the outer surface 653s of the extending portion 653 of the tubular member 650 in order to form a smooth surface continuous with the outer surface 40s of the second proximal portion 40b of the distal side coating layer 40.

Thereafter, similarly to the above-described first embodiment, the swaging is further performed. As illustrated in FIG. 16(C), a tapered shape whose outer diameter gradually decreases toward the proximal side is formed in the proximal portion 652 of the tubular member 650. Concurrently with the processing for reducing the diameter of the lumen of the tubular member 650, the tapered shape of the proximal portion 652 may be formed.

Finally, the guide wire 610 is obtained by joining the first core portion 21 to the second core portion 22 having the proximal side coating layer 60 formed therein.

The guide wire 610 and the manufacturing method of the guide wire 610 according to the third embodiment can also achieve an advantageous effect the same as that according to the above-described first embodiment.

In addition, the distal portion 651 of the tubular member 650 has the extending portion 653 which extends to the distal side so as to cover at least a portion (first proximal portion 40a) of the proximal portion of the distal side coating layer 40. In this manner, when the catheter to which the guide wire 610 is inserted moves from the proximal side of the tubular member 650 to the distal side, the first proximal portion 40a of the distal side coating layer 40 can be more reliably prevented from getting caught on the catheter. In addition, the compressive bonding surface 70s is formed between the inner surface of the extending portion 653 and the outer surface 40s of the first proximal portion 40a of the distal side coating layer 40. Accordingly, an area of the compressive bonding surface 70s further increases, and the first proximal portion 40a of the distal side coating layer 40 can be pressed inward in the radial direction. Therefore, the tubular member 650 is more firmly fixed to the distal side coating layer 40. As a result, the tubular member 650 can be further prevented from pulling away from the core portion 20. In this manner, the tubular member 650 can be prevented from slipping out of the core portion 20, and the tubular member 650 can be restrained from damaging the inner wall inside the body lumen or getting caught on the catheter.

In addition, as in the present embodiment, in a case where the outer surface 653s of the extending portion 653 of the tubular member 650 includes the surface continuous with the outer surface 40s of the second proximal portion 40b of the distal side coating layer 40, when the guide wire 610 is inserted into the body lumen or the lumen of the catheter, the guide wire 610 can be prevented from getting caught on the inner wall inside the body lumen or the catheter.

Next, modification examples of the above-described third embodiment will be described. In the description of the modification examples, the same reference numerals will be given to configurations the same as those according to the above-described third embodiment, and description thereof will be omitted. In addition, points particularly not described in each modification example can be configured similarly to the above-described third embodiment.

### Modification Example 1 of Third Embodiment

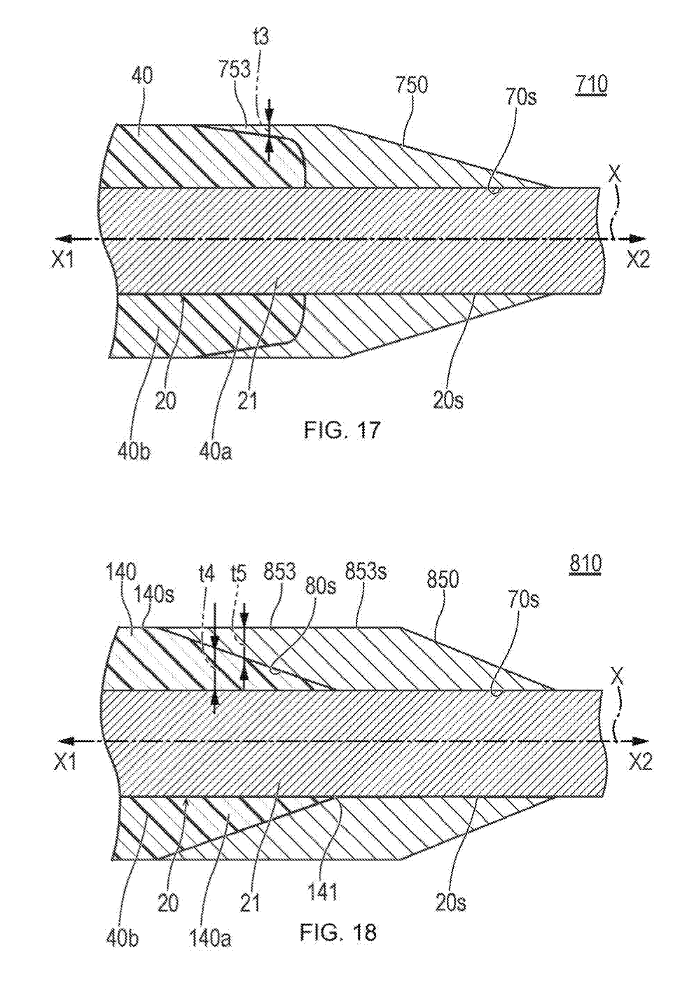

FIG. 17 is an axial cross-sectional view illustrating an enlarged main portion of a guide wire 710 according to Modification Example 1 of the third embodiment.

As illustrated in FIG. 17, unlike in the above-described third embodiment, the guide wire 710 according to the Modification Example 1 of the third embodiment is formed in a tapered shape in which a thickness t3 of the extending portion 753 of the tubular member 750 gradually decreases toward the distal side. A manufacturing method of the guide wire 710 is the same as that according to the above-described third embodiment. The guide wire 710 and the manufacturing method of the guide wire 710 according to Modification Example 1 of the third embodiment can also achieve an advantageous effect the same as that according to the above-described third embodiment.

In addition, the thickness t3 of the extending portion 753 of the tubular member 750 gradually decreases toward the distal side. Accordingly, the rigidity of the guide wire 710 can be gradually changed. Therefore, it is possible to restrain a sudden change in the rigidity. As a result, the followability of the guide wire 710 following the blood vessel can be improved, and the guide wire 710 can be prevented from being bent.

### Modification Example 2 of Third Embodiment

FIG. 18 is an axial cross-sectional view illustrating an enlarged main portion of a guide wire 810 according to Modification Example 2 of the third embodiment.

As illustrated in FIG. 18, in the guide wire 810 according to Modification Example 2 of the third embodiment, a thickness t4 of the first proximal portion 140a which is a portion covered with the extending portion 853 of the tubular member 850 in the proximal portion of the distal side coating layer 140 is formed in a tapered shape which gradually decreases toward the proximal side up to the proximal end 141. In addition, the extending portion 853 of the tubular member 850 is formed in a tapered shape in whose thickness t5 gradually decreases toward the distal side so as to extend along the tapered shape of the first proximal portion 140a of the distal side coating layer 140.

A manufacturing method of the guide wire 810 is the same as that according to the above-described third embodiment. The guide wire 810 and the manufacturing method of the guide wire 810 according to Modification Example 2 of the third embodiment can also achieve an advantageous effect the same as that according to the above-described third embodiment.

In addition, the extending portion 853 of the tubular member 850 is formed in the tapered shape so as to extend along the tapered shape of the first proximal portion 140a of the distal side coating layer 140. Accordingly, it is possible to increase a contact area of a contact surface 80s between the outer surface of the first proximal portion 140a of the distal side coating layer 140 and the inner surface of the tubular member 850. In this manner, it is possible to further improve a function to prevent the distal side coating layer 140 from being separated and stripped off from the core portion 20.

### Alternative Example

Hereinafter, alternative examples according to the present invention will be described.

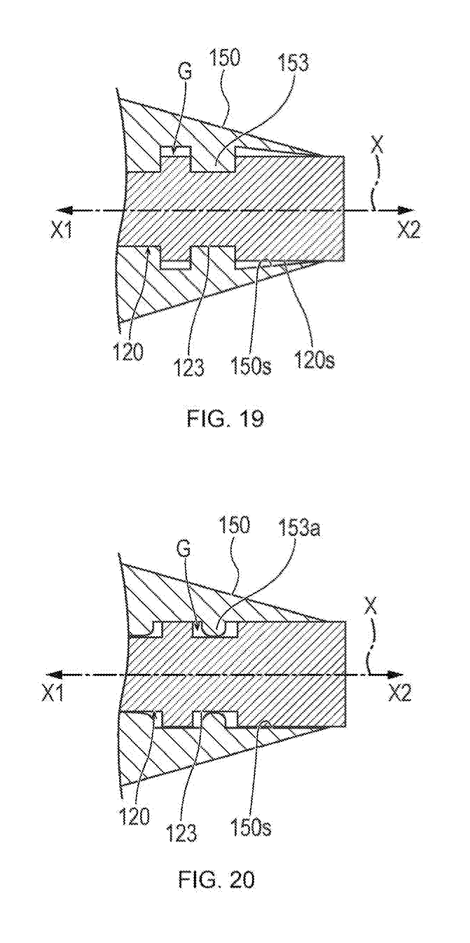

In the above-described embodiments and modification examples, a form in which the compressive bonding surface 70s is formed on the entire inner surface of the tubular member has been described as a preferred form. However, as long as the compressive bonding surface 70s is formed in at least a portion of the inner surface of the tubular member, the present disclosure is not limited to the above-described form. For example, as illustrated in FIG. 19, the configuration may have the gap G between the inner surface 150s of the tubular member 150 and the outer surface 120s of the core portion 120 according to the second embodiment.

In addition, the shape of the engagement portion of the core portion and the shape of the engagement pairing portion of the tubular member are not particularly limited as long as the engagement portion of the core portion has a shape capable of engaging with the engagement pairing portion of the tubular member. In addition, the present disclosure is not limited to a form in which the shape of the engagement portion 123 of the core portion 120 and the shape of the engagement pairing portion 153 of the tubular member 150 coincide with and are fitted to each other as in the second embodiment. For example, as illustrated in FIG. 20, the shape of the engagement pairing portion 153a of the tubular member 150 may be formed to be smaller than the shape of the engagement portion 123 of the core portion 120 so as to have the gap G.

In addition, in the second embodiment and the modification examples thereof, a form has been described in which the engagement portion of the core portion includes the concave portion, and the engagement pairing portion of the tubular member includes the convex portion. However, the present disclosure is limited thereto. The present disclosure may adopt a form in which the engagement portion of the core portion includes the convex portion, and the engagement pairing portion of the tubular member includes the concave portion.

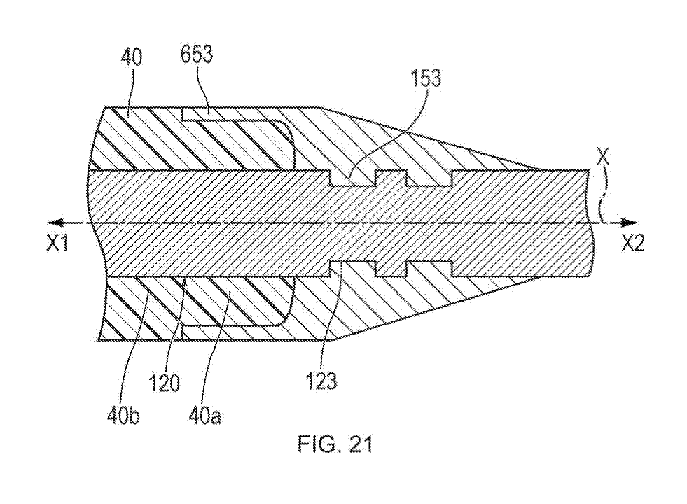

In addition, the configurations of the core portion and the tubular member which are described above in the embodiments and modification examples can be appropriately and selectively combined with each other so as to provide one guide wire. For example, as illustrated in FIG. 21, a configuration in which the core portion 120 described in the second embodiment includes the engagement portion 123, a configuration in which the tubular member includes the engagement pairing portion 153, and a configuration in which the tubular member described in the third embodiment includes the extending portion 653 which covers the proximal portion of the distal side coating layer may be combined with each other.

In addition, in the above-described embodiments and modification examples, a configuration has been described in which the tubular member has a ring shape. However, for example, the tubular member may be formed so that an internally and externally communicating slit is formed in all regions in the axial direction thereof, that is, so that the axially orthogonal cross-sectional shape may be a C-shape.

In addition, the shape and the configuration except for the main portion of the guide wire are not limited to the configurations described above with reference to the embodiments, the modification examples, and the accompanying drawings in the present specification. A shape and a configuration of the known guide wire can be used. For example, a configuration may be adopted which does not include the coil portion, or a radiopaque marker may be appropriately disposed in the distal portion.

The detailed description above describes features and aspects of embodiments of a guide wire and manufacturing method disclosed by way of example. The invention is not limited, however, to the precise embodiments and variations described. Changes, modifications and equivalents can be employed by one skilled in the art without departing from the spirit and scope of the invention as defined in the appended claims. It is expressly intended that all such changes, modifications and equivalents which fall within the scope of the claims are embraced by the claims.

What is claimed is:

1. A guide wire comprising:
an elongated core portion;
a resin coating layer that is made of a resin material, and that covers a distal portion of the core portion; and
a metallic tubular member that has a lumen into which the core portion is inserted, and that is disposed so as to be in contact with at least a portion of a proximal end of the resin coating layer,
wherein the tubular member is formed in a state where the lumen is reduced in diameter by cold forging, and at least a portion of an inner surface forming the lumen has a compressive bonding surface which is compressively bonded to an outer surface of the core portion.

2. The guide wire according to claim 1,
wherein the compressive bonding surface of the tubular member is formed over the entire inner surface of the tubular member.

3. The guide wire according to claim 1,
wherein the core portion has at least one engagement portion which engages with the tubular member, and
wherein the tubular member has at least one engagement pairing portion which corresponds to and engages with the engagement portion.

4. The guide wire according to claim 3,
wherein the engagement portion is disposed in at least a portion of the outer surface of the core portion, and includes a concave portion which is recessed inward in a radial direction in a concave shape, and
wherein the engagement pairing portion is disposed in at least a portion of the inner surface of the tubular member, and includes a convex portion which protrudes to the core portion side in a convex shape.

5. The guide wire according to claim 1,
wherein an outer surface of a distal portion of the tubular member is continuous with an outer surface of the resin coating layer.

6. The guide wire according to claim 1,
wherein a proximal portion of the tubular member has a tapered shape whose outer diameter gradually decreases toward a proximal side.

7. The guide wire according to claim 1,
wherein a distal portion of the tubular member has an extending portion which extends to a distal side so as to cover at least a portion of a proximal portion of the resin coating layer.

8. A guide wire comprising:
an elongated core having a concave portion which is recessed inward in a radial direction in a concave shape;
a resin coating layer that is made of a resin material, and that covers a distal portion of the core; and
a metallic tubular member that has a lumen into which the core is inserted, and that is disposed so as to be in contact with at least a portion of a proximal end of the resin coating layer, and that has a convex portion which protrudes toward the core in a convex shape and engages the concave portion.

9. The guide wire according to claim 8,
wherein an outer surface of a distal portion of the tubular member is continuous with an outer surface of the resin coating layer.

10. The guide wire according to claim 8,
wherein a proximal portion of the tubular member has a tapered shape whose outer diameter gradually decreases toward a proximal side.

11. The guide wire according to claim 8,
wherein a distal portion of the tubular member has an extending portion which extends to a distal side so as to cover at least a portion of a proximal portion of the resin coating layer.

12. A manufacturing method of a guide wire which has an elongated core portion, a resin coating layer made of a resin material so as to cover a distal portion of the core portion, and a metallic tubular member, the method comprising:
a step of inserting the core portion into a lumen of the tubular member, and disposing the tubular member so as to be in contact with at least a portion of a proximal end of the resin coating layer; and
a cold forging step of reducing a diameter of the lumen of the tubular member by cold forging, and forming a compressive bonding surface compressively bonded to an outer surface of the core portion in at least a portion of an inner surface forming the lumen.

13. The manufacturing method of a guide wire according to claim 12,
wherein in the cold forging step, a compressive force toward the core portion side is applied to the tubular member, and the diameter of the lumen of the tubular member is reduced by compressing the tubular member to the core portion side.

14. The manufacturing method of a guide wire according to claim 12,
wherein in the cold forging step, the compressive bonding surface is formed over the entire inner surface of the tubular member.

15. The manufacturing method of a guide wire according to claim 12, further comprising:
a step of forming at least one engagement portion capable of engaging with the tubular member in the core portion before the cold forging step,
wherein in the cold forging step, at least one engagement pairing portion which corresponds to and engages with the engagement portion is formed.

16. The manufacturing method of a guide wire according to claim 12,
wherein in the cold forging step, an outer surface of a distal portion of the tubular member is formed to serve as a surface continuous with an outer surface of the resin coating layer.
